(19) United States
(12) Patent Application Publication
(10) Pub. No.: US 2009/0165801 A1
(43) Pub. Date: Jul. 2, 2009

(54) CARBON DIOXIDE DETECTOR HAVING AN ACRYLIC BASED SUBSTRATE

(75) Inventor: Rafael Ostrowski, Pittsburg, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/338,786

(22) Filed: Dec. 18, 2008

Related U.S. Application Data
(60) Provisional application No. 61/009,680, filed on Dec. 31, 2007.

(51) Int. Cl. A61M 16/04; A61B 5/08; A61M 16/00

(52) U.S. Cl. 128/205.23; 600/532; 128/207.15

(57) ABSTRACT

Embodiments disclosed herein may include a carbon dioxide detector having an acrylic-based substrate impregnated with a carbon dioxide sensitive dye. The carbon dioxide sensitive dye may include a pH indicator which changes color based on the concentration of carbon dioxide in the air at the surface of the indicator. In some embodiments, the impregnated polymer may be incorporated into a window in the carbon dioxide detector, while in other embodiments an element of a ventilation system may be composed of the impregnated polymer. For example, a casing for an endotracheal tube or the tube itself may be composed of the acrylic-based polymer impregnated with the carbon dioxide sensitive dye.

### RELATED APPLICATION

This application claims priority from U.S. Patent Application No. 61/009,680 which was filed on Dec. 31, 2007, and is incorporated herein by reference in its entirety.

### BACKGROUND

The present disclosure relates to a carbon dioxide detector having an acrylic based substrate.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Respiratory gasses may be readily distinguished from non-respiratory gasses by carbon dioxide content. Exhaled respiratory gas in a human typically contains between 3% and 5% carbon dioxide. In contrast, ambient air has only approximately 0.03% carbon dioxide. Normal esophageal gas has similarly low levels of carbon dioxide.

The detection of respiratory gasses via carbon dioxide content may be useful in a variety of circumstances. For example, one may determine whether an endotracheal tube has been correctly placed in the trachea rather than in the esophagus by detecting the presence of carbon dioxide in air exiting the tube. If carbon dioxide levels consistent with respiration are present, then the tube is correctly placed in the trachea. If only low carbon dioxide levels consistent with placement in the esophagus are present, then the tube may have been incorrectly placed and may need to be removed and reinserted correctly. Additionally, if a tracheal tube is present in the trachea, but carbon dioxide levels in respired gas are low, this may be indicative of perfusion failure.

Continued detection of carbon dioxide in respired gas may also be useful in determining if an endotracheal tube has been dislodged or if the cuff is deflated or incorrectly inflated, and if breathing and perfusion continue to be normal. Current products can detect carbon dioxide in respired air using various chemicals sensitive to the presence of carbon dioxide on a substrate such as a cellulose filter paper, for example Whatman paper. However, the substrate of such products may have a relatively short active lifetime due to the hydrophilic nature to absorb water from respiration. Additionally, the substrate of such products may have a relatively short inactive lifetime due to the formation of acidic and oxidative substances to which the dye may react.

### BRIEF DESCRIPTION OF THE DRAWINGS

A more complete understanding of the present disclosure thereof may be acquired by referring to the following description taken in conjunction with the accompanying drawings. These drawings represent only certain embodiments of the present disclosure.

FIG. 1 illustrates a colorimetric carbon dioxide detector in accordance with embodiments of the present disclosure;

FIG. 2 illustrates a carbon dioxide detector systems in accordance with embodiments of the present disclosure;

FIG. 3 illustrates another carbon dioxide detector system in accordance with embodiments of the present disclosure;

FIG. 4 illustrates the carbon dioxide detector system of FIG. 3 coupled to a resuscitator in accordance with embodiments of the present disclosure; and

FIG. 5 illustrates a carbon dioxide detecting endotracheal tube in accordance with embodiments of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In accordance with the present disclosure, there may be provided a carbon dioxide (CO₂) detector having an acrylic polymer substrate. More specifically, the acrylic polymer may be impregnated with a pH sensitive dye prior to, during, or after polymerization such that the dye may be distributed substantially throughout the polymer. In previous carbon dioxide detectors, the dye was added to the substrate via an indicator solution (i.e., the substrate was soaked in the solution). The pH sensitive dye may act as an indicator which changes color at a different pH. Accordingly, the detector may change colors in the presence of different concentrations of carbon dioxide.

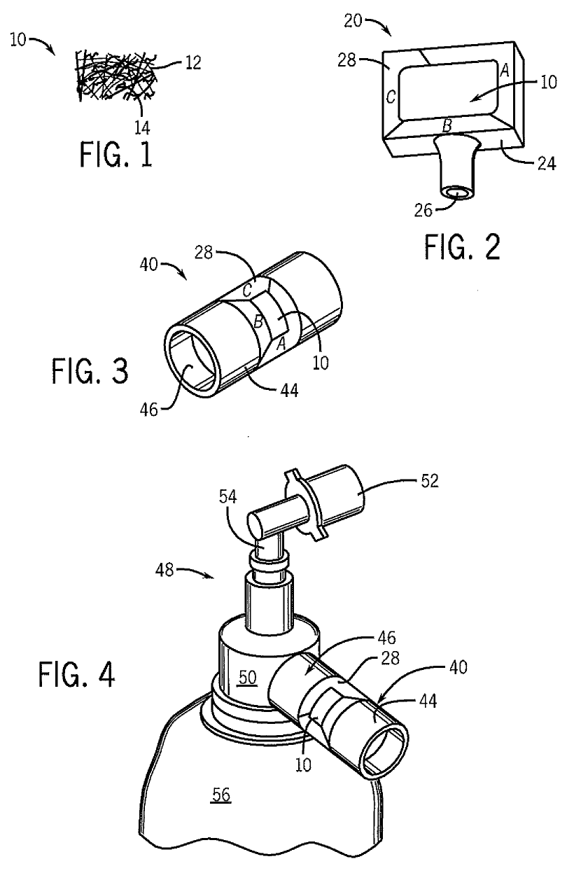

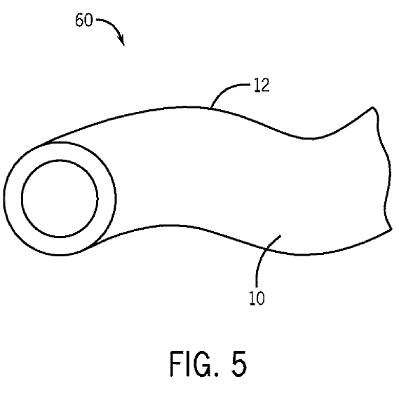

In an embodiment illustrated in FIG. 1, a carbon dioxide detector 10 may include a substrate 12 and an indicator 14, such as a pH sensitive dye. The detector 10 may be sized appropriately for use in a detector system, such as those shown in FIGS. 2 and 3. In addition, the detector 10 may be incorporated into and/or used to form an endotracheal tube, as shown in FIG. 5.

The substrate 12 may include an acrylic polymer. Specifically, it may include a polymer made of acrylic based monomers, such as, for example, sodium acrylate, acrylic acid, acrylonitrile, acrylamide, methyl acrylate, methylmethacrylate, aliphatic acrylates, and so forth. Polymerization of the substrate 12 may occur by emulsion, dispersion, suspension, or solution polymerization. The substrate 12 may be a porous polymer permeable to carbon dioxide to facilitate rapid infiltration of carbon dioxide gas therein.

In an embodiment, the acrylic polymer substrate 12 may have a higher hydrophobicity than cellulose paper substrates, thereby providing a more basic environment for the indicator 14. Such a basic environment may increase the color sensitivity of the indicator 14 in a low carbon dioxide environment, such as less than 0.5%. Acrylic is an electron rich compound, and therefore is a good Bronstead and Lewis base. The resulting ability to accept protons from proton rich compounds and to donate a pair of electrons to electron poor compounds enables the indicator 14 to remain unaffected by acidic and oxidative compounds. Enough carbonic acid may be formed to affect the indicator, as described below, to overcome any neutralization by the acrylic substrate 12.

In order to adjust the hydrophilicity and nucleophilisity of the substrate 12, monomers having certain properties may be included in the polymerization reaction. In an embodiment, enough hydrophilic monomers may be incorporated into the polymerization of the substrate 12 to enable fast carbon dioxide solvation and desolvation within the substrate 12 without retaining so much water that the efficiency of the detector 10 is degraded. Similarly, nucleophilic monomers (e.g., hydroxyalkyl methacrylate, acrylamide, aminoalkyl methacrylate, and so forth) may be incorporated into the polymerization reaction to provide the desired nucleophilisity for the carbonic acid reaction. Furthermore, the polymer substrate 12 may incorporate other monomers, such as styrene and vinylene, to strengthen and/or modify other properties of the material depending on the application.

In addition, various pH sensitive indicators 14 may be used in the indicator solution 14. These include, but are not limited to, metacresol purple, thymol blue, cresol red, phenol red, xylenol blue, a 3:1 mixture of cresol red and thymol blue, bromothymol blue, neutral red, phenolphthalein, rosolic acid, α-naphthelphthalein, and orange I. Other pH indicators 14, the color change that occurs, and the relevant pH as well as other information may be found in the CRC Handbook of Chemistry and Physics, 8-17, 75th Edition 1994.

In an embodiment, the indicator 14 may be impregnated into the substrate 12. The impregnation may occur before, during, or after the polymerization reaction. Because the indicator 14 is impregnated into the polymer substrate 12, it is not removed to any substantial degree from the substrate during reaction with water. Indeed, even if placed in liquid water, the polymer 12 generally prevents elution of the indicator 14. The indicator 14 may be disposed throughout the bulk of the substrate 12, including on its surface. Therefore, in certain embodiments, the pH indicator 14 may measure the pH of the bulk substrate 12.

The carbon dioxide detector 10 may operate by changing colors based on the concentration of carbon dioxide in the air. For example, in one embodiment, the carbon dioxide detector 10 may be utilized to detect the concentration of carbon dioxide present in air respired from a human. The pH sensitive dye 14 enables this detection by changing colors based on the pH of the dye 14. Carbon dioxide in the air may react with water in the presence of a nucleophile, as described by the following reversible equations:

CO₂+H₂O [symbol] HCO₃⁻+H⁺, (1)

CO₂+H₂O [symbol] CO₃²⁻+2H⁺, and (2)

CO₂+R₂NH [symbol] R₂NCOO⁻+H⁺. (3)

As can be seen, these reactions produce a carbonate, bicarbonate, or carbamate moiety and hydrogen ions (H⁺). When a certain concentration of H⁺ is reached, the pH indicator changes color. Water and basisity for the above reactions may be provided by the polymer substrate, as described above.

Reactions (1)-(3) may deplete the hydroxyl ion (OH⁻) or amine at an interface between the indicator 14 and air, thereby lowering the pH at the surface of the substrate 12 where the indicator 14 is adjacent or nearly adjacent to air. This depletion may result in the diffusion of base from the polymer substrate 12 to the indicator 14 to maintain a surface pH similar to that of the substrate 12 overall.

More specifically, the concentration of OH⁻ or amine in the bulk of the substrate 12 may affect the rate of diffusion of base to the surface of the substrate 12. The rate of the chemical reaction at this surface is determined by the nature of each specific reacting species. For example, the rate of reaction at the surface of the substrate 12 may be expressed by the following equation:

R═K_{A}[CO₂][A], (4)

where [x] represents the concentration of a species in moles/liter, and K_{A} is a constant specific for the reactant species A. In a specific embodiment, A is the indicator.

In an embodiment, the balance of base between the surface and the remainder of the substrate 12 may be influenced by the contact time between the surface and the gas to which it is exposed; the composition of the substrate 12, which determines the diffusivity constant for species A and thus the rate of diffusion of species A to the surface; and the concentration of carbon dioxide in the gas, which determines the rate of diffusion of carbon dioxide into or near the surface of the substrate 12 where it may react with the indicator 14.

Certain elements of Reaction (4) may be adjusted based on the manner in which the carbon dioxide detector 10 is constructed and used. For example, the concentration of OH⁻ or amine in the substrate 12, the rate of the chemical reaction, the contact time between the indicator surface and the gas, and the diffusivity constant for species A may be set. Accordingly, the concentration of carbon dioxide in the gas is the only variable parameter with significant effect, enabling its measurement.

In an embodiment, the concentration of OH⁻ or amine in the substrate 12 and the rate of the chemical reaction may be adjusted such that the pH near the surface of the substrate 12 decreases sufficiently in the presence of a certain concentration of carbon dioxide to cause a color change in the indicator 14. For example, the color change may occur if the concentration of carbon dioxide in the tested air is greater than approximately 2%. This color change may occur within 1 to 20 seconds of exposure of carbon dioxide detector 10 to the air In a specific example, a concentration of OH⁻ sufficient to produce a pH of 9.6±0.2 in the substrate 12 is sufficient to provide this sensitivity.

In an embodiment, the carbon dioxide detector 10 may be configured such that the indicator 14 is exposed to or near air or gas within the detector 10. More specifically, the indicator 14 may be able to respond to concentrations of carbon dioxide normally present in air respired from a human, such as between approximately 2% and 5% or higher. The indicator 14 may also be able to respond to concentrations of carbon dioxide in air respired from a human with perfusion failure, such as concentrations between approximately 0.5% and 2%. Finally, the indicator 14 may show no response to carbon dioxide concentrations normally present in external air or esophageal air, such as concentrations below approximately 0.5% and more specifically, concentrations between 0.03% and 0.5%.

In an embodiment, indicator response may include a colorimetric indication, such as a change of the indicator from one color to a distinct second color. Once the color begins to change, the change from one color to the other color may be virtually instantaneous as seen by the human eye. As discussed above, the indicator 14 may show no response to ordinary levels of carbon dioxide in the air. Therefore, the detector 10 may be removed from packaging and connected to another device, such as a resuscitator, without degrading. Exposure to air may cause the pH of the indicator 14 to gradually decrease, but if such decrease is sufficiently slow, a desired minimum time period without color change may still be achieved.

Use of the acrylic polymer substrate 12 may result in desirable response time and shelf life for the carbon dioxide detector 10, while retaining the capacity of the detector 10 to cycle from one color to another quickly from breath to breath. For example, in some carbon dioxide detectors, reaction of the substrate with cresol red, which is used as a color indicator, eventually changes the color indicator irreversibly from purple to yellow. This change makes the detector color insensitive to the presence or absence of carbon dioxide. As a result, the detector system is no longer functional. Although packaging can help prevent sensor aging, it nevertheless may limit shelf life. Acrylic substrates do not react with cresol red. As a result, the same shelf life obtained with other substrates may be achieved with the acrylic polymer substrate 12 using a more cost effective packaging, or a longer shelf life in the same packaging may be achieved.

In certain embodiments, the shelf life of the carbon dioxide detector 10 may be greater than 5 years, greater than 10 years, or greater than 14 years. Further, while the shelf life of the carbon dioxide detector 10 may be greatly improved, the packaging employed may be reduced, due to the stability of the acrylic-based carbon dioxide detector 10. While other calorimetric carbon dioxide detection systems may employ dessicants to extend their shelf lives, the acrylic-based carbon dioxide detector 10 may achieve a long shelf life (e.g. several years) without the use of a dessicant.

In an embodiment, the substrate 12 may exhibit an improved color cycling pattern in the presence of carbon dioxide. For example, with use of metacresol purple in the indicator solution 14, the substrate 12 may change from a deep purple to a light tan color, rather than purple to yellow, in the presence of carbon dioxide. One advantage of a purple-to-tan color change rather than a purple-to-yellow color change is that the contrast ratio between purple and tan is particularly advantageous, allowing a healthcare worker to distinguish finer gradations of carbon dioxide levels. Further, the purple-to-tan color change is also helpful for people with color blindness, which most often impairs acuity in the green-yellow-red portion of the spectrum.

In an embodiment, the performance of carbon dioxide detectors in humid air is significant to clinical use because exhaled breath contains considerable amounts of water. Thus, performance in humid conditions is indicative of performance with actual patients. It may affect the use-life of a detector. Accordingly, the carbon dioxide detector 10 having the acrylic substrate 12 may show faster breath-to-breath response than one having a cellulose fiber substrate such as paper. This faster response may also be facilitated by the highly porous nature of the acrylic polymer substrate 12, which enables easier penetration of carbon dioxide than does a cellulose fiber substrate. This may indicate a longer use-life of the acrylic-based detector 10.

In an embodiment, shown in FIG. 2, a detector system 20 may include the carbon dioxide detector 10, a housing 24, an air intake 26, and color indicators 28. The detector system 20 may be configured to fit into a further system, such as a resuscitator and/or ventilation system. The further system may supply air to the detector system 20 for measurement. Specifically, the further system may be connected to the respiratory pathway of a patient.

Parts of the detector system 20, such as the housing 24 and/or the air intake 26 may be made from a rigid material. For example, they may be made from a plastic, such as a clear, colorless, transparent plastic. By way of further example, the housing 24 and/or the air intake 26 may be made from polyethylene, polypropylene, an acrylic polymer such as PLEXIGLAS® polymer, polycarbonate, nylon, polysytrene, styrene-acrylonitrile copolymer, or combinations thereof. In some embodiments, the detector 10 may be a piece of acrylic disposed within the detector 20 and visible through a clear window 30. In other embodiment, the acrylic polymer substrate 12 impregnated with the indicator 14 may be utilized to form the housing 24 and/or the air intake 26.

The air intake 26 may also serve to couple the detector system 20 to the further system. The air intake 26 may be releasably secured to the housing 24, such as by a threaded engagement, or it may form an integral unit with the housing 24. In addition, the air intake 26 may have a threaded engagement, tab, grooves, or other features to allow it to be releasably secured to the further system. For example, a pressure fit may be used to couple the detector system 20 to a manual resuscitator, such as the INdGO® manual disposable resuscitator available from Nellcor Puritan Bennett LLC and/or Covidien.

The color indicators 28 may approximately match the color of the indicator 14 in the presence of different levels of carbon dioxide. Additionally, the color indicators 28 may include written or other visual information to allow a user to determine what carbon dioxide concentrations are indicated by various colors. For example, region A may show one or various shades that correlate with a low carbon dioxide concentration, such as below approximately 0.5% or between approximately 0.03% and 0.5%. In an embodiment, region A may contain shades of purple. Region C may show one or various shades that correlate with a high carbon dioxide concentration typical of respired air, such as above approximately 2% or between 2% and 5%. In an embodiment, region C may contain shades of yellow or tan. Optional region B may indicate carbon dioxide concentrations above that of normal or esophageal air, but below that corresponding with normal respiration. For example, region B may indicate carbon dioxide concentrations common in respired air of a patient suffering from perfusion failure. Region B may show one or various shades that correlate with carbon dioxide concentrations of between approximately 0.5% and 2%. In an embodiment, region B may contain shades of grayish purple.

In another embodiment, shown in FIG. 3, a detector system 40 may include the carbon dioxide detector 10, a housing 44, an air intake 46, and the color indicators 28. The housing 44 and the air intake 46 may be similar to the housing 24 and the air intake 26 in composition and function. However, they may be of a different shape to allow use with other systems.

In an embodiment of the disclosure, shown in FIG. 4, a resuscitator 48 may include the carbon dioxide detector system 40 attached to a resuscitator housing 50. The carbon dioxide detector systems 16 and 20 may also be used with the resuscitator 48. The resuscitator 48 may also have an endotracheal tube attachment 52, a swivel joint 54, and a bag 56. The resuscitator 48 may be formed in any manner known in the art. In particular, the resuscitator 48 may be the INdGO™ disposable manual resuscitator.

Carbon dioxide detection may include in-stream detection, as in the EasyCap™ system available from Nellcor Puritan Bennett LLC and/or Covidien. Detection may also include “side-stream” detection, as in the INdCAP® system available from Nellcor Puritan Bennett LLC and/or Covidien. The detection system may be modified to facilitate either or any form of detection.

In an embodiment of the present disclosure, shown in FIG. 5, an endotracheal tube 60 may be composed of the substrate 12. In this embodiment, all or a portion of the tube 60 may contain the indicator 14 impregnated therein. A hydrophobic coating may be applied to the exterior of the tube 60 to prevent interference from ambient carbon dioxide and water. The tube 60 may change colors depending on the concentration of carbon dioxide present in the air flowing therethrough. In this embodiment, fewer items are needed to determine the correct placement of the endotracheal tube 60 within the patient's trachea because the tube 60 itself is the detector 10.

Manufacturing of the carbon dioxide detector 10 may include formation of the substrate 12 and impregnation of the indicator 14. The indicator 14 may be incorporated into the polymer substrate 12 before, during, or after the polymerization reaction. The acrylic polymer 12 impregnated with the indicator 14 may be formed into any desired configuration for the detector 10. For example, the detector 10 may be a rectangular piece of the polymer 12, as illustrated in FIG. 2, or it may be the endotracheal tube 60, as illustrated in FIG. 5.

The carbon dioxide detector 10 may then be incorporated into a detector system, for example, such as those shown in FIGS. 2 and 3. The detector system may be packaged in protective packaging or incorporated in a further system, such as the resuscitator 48, before packaging. During its formation and handling prior to packaging, the carbon dioxide detector 10 may be kept in conditions to minimize or control chemical reactions that might negatively influence its reliability. The carbon dioxide detector 10 of the present disclosure may require less stringent pre-packaging conditions than current cellulose filter paper detectors because of improvements in resistance to negative effects of humidity and room air. Carbon dioxide detectors, detection systems, of further systems may also be created in a sterile or clean environment or later sterilized.

In use, the carbon dioxide detector 10 may be deposited within an air pathway. The air may infiltrate the substrate 12, and any carbon dioxide in the air reacts with water bonded to the hydrophilic elements of the substrate 12. The water may also be absorbed from the air. Depending on the concentration of carbon dioxide in the air, the air may produce a color change in the indicator 14. In certain embodiments, the carbon dioxide detector 10 may specifically be used to detect air from an endotracheal tube. The presence of carbon dioxide may indicate proper placement of the tube in the trachea of a patient rather than in the esophagus. Accordingly, the carbon dioxide detector 10 may be utilized to detect incorrect placement in sufficiently little time to allow removal of the tube and placement in the trachea before the patient suffers serious injury or death. If the indicator 10 changes color back and forth between a low carbon dioxide color and a high color dioxide color, this may indicate that the patient is breathing normally. Alternatively, if the indicator 10 changes to a color indicating low concentrations of carbon dioxide still above concentrations in the air, this may indicate a perfusion failure in the patient.

The carbon dioxide detector 10 may be used to monitor any patient benefiting from an endotracheal tube or other endotracheal system, e.g. a resuscitator fitted with a mask. More specifically, it may be used to monitor a human patient, such as a trauma victim, an anesthetized patient, a cardiac arrest victim, a patient suffering from airway obstruction, or a patient suffering from respiratory failure.

While embodiments of this disclosure have been depicted, described, and are defined by reference to specific example embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure. For example, the substrate may be formed in a variety of ways; various indicators, alkali sources and other components may be used in the indicator solution; the indicator solution may be placed on the substrate in a variety of ways; multiple indicators may be used to detect narrower ranges of carbon dioxide concentration; and the system may take a variety of shapes.

What is claimed is:

1. A carbon dioxide detector, comprising:
an acrylic polymer substrate; and
a carbon dioxide sensitive dye generally impregnated within the acrylic polymer substrate.

2. The carbon dioxide detector of claim 1, wherein the acrylic polymer substrate comprises a polymer of sodium acrylate, acrylic acid, acrylonitrile, acrylamide, methyl acrylate, methylmethacrylate, aliphatic acrylates, hydroxyalkyl methacrylate, acrylamide, aminoalkyl methacrylate, styrene, and/or vinylene, and/or combinations thereof.

3. The carbon dioxide detector of claim 1, wherein the carbon dioxide sensitive dye comprises a pH indicator, including metacresol purple, thymol blue, cresol red, phenol red, xylenol blue, bromothymol blue, neutral red, phenolphthalein, rosolic acid, α-naphthelphthalein, and/or orange I, and/or combinations thereof.

4. The carbon dioxide detector of claim 1, wherein the acrylic polymer substrate contains enough hydrophilic elements to enable acidification of carbon dioxide gas.

5. The carbon dioxide detector of claim 1, wherein the carbon dioxide sensitive dye is impregnated within the acrylic polymer substrate during polymerization.

6. The carbon dioxide detector of claim 1, comprising a plastic element capable of being placed in a visible location of a ventilation system.

7. An endotracheal tube being made of an acrylic polymer having a carbon dioxide sensitive dye disposed therein, wherein the endotracheal tube is capable of changing color in response to a concentration of carbon dioxide in the tube.

8. The endotracheal tube of claim 7, wherein the acrylic polymer comprises a polymer of sodium acrylate, acrylic acid, acrylonitrile, acrylamide, methyl acrylate, methylmethacrylate, aliphatic acrylates, hydroxyalkyl methacrylate, acrylamide, aminoalkyl methacrylate, styrene, and/or vinylene, and/or combinations thereof.

9. The carbon dioxide detector of claim 7, wherein the acrylic polymer contains enough hydrophilic elements to enable acidification of carbon dioxide gas.

10. The carbon dioxide detector of claim 7, wherein the carbon dioxide sensitive dye comprises a pH indicator, including metacresol purple, thymol blue, cresol red, phenol red, xylenol blue, bromothymol blue, neutral red, phenolphthalein, rosolic acid, α-naphthelphthalein, and/or orange I, and/or combinations thereof.

11. A ventilation system, comprising:
a resuscitator; and
a carbon dioxide detector operably coupled to the resuscitator and comprising an acrylic polymer substrate generally impregnated with a carbon dioxide sensitive dye.

12. The ventilation system of claim 11, wherein the carbon dioxide sensitive dye is capable of changing color depending on a carbon dioxide concentration in air.

13. The ventilation system of claim 11, wherein the carbon dioxide detector comprises an endotracheal tube.

14. The ventilation system of claim 11, wherein the carbon dioxide detector comprises a generally transparent window through which the carbon dioxide sensitive dye may be observed.

15. The ventilation system of claim 11, wherein the carbon dioxide detector comprises a color comparison portion configured to enable determination of the carbon dioxide concentration by comparison to the color of the carbon dioxide sensitive dye.

16. The ventilation system of claim 11, wherein the carbon dioxide sensitive dye comprises a pH indicator, including metacresol purple, thymol blue, cresol red, phenol red, xylenol blue, bromothymol blue, neutral red, phenolphthalein, rosolic acid, α-naphthelphthalein, and/or orange I, and/or combinations thereof.

17. A method comprising:
providing a carbon dioxide detector having a color-changing element and a color comparison element, wherein the color-changing element comprises:
an acrylic polymer substrate; and
a carbon dioxide sensitive dye generally impregnated within the acrylic polymer substrate.

18. The method of claim 17, comprising coupling the carbon dioxide detector to a resuscitator.
